(19) United States
(12) Patent Application Publication
(10) Pub. No.: US 2016/0176855 A1
(43) Pub. Date: Jun. 23, 2016

(54) Novel Sterically Hindered Cyclic Amines

(71) Applicant: CLARIANT INTERNATIONAL LTD, Muttenz (CH)

(72) Inventors: Chrisoph KRÖHNKE, Nandlstadt (DE); Axhim KOCH, Moosburg (DE)

(73) Assignee: CLARIANT INTERNATIONAL LTD., Muttenz (CH)

(21) Appl. No.: 14/907,789

(22) PCT Filed: Jul. 25, 2014
(86) PCT No.: PCT/EP2014/002034

§ 371 Date: Jan. 26, 2016

(30) Foreign Application Priority Data
Jul. 30, 2013 (DE) 10 2013 012 622.3

(51) Int. Cl. C07D 405/14; C08K 5/3462; C08K 5/3435

(52) U.S. Cl. CPC C07D 405/14; C08K 5/3435; C08K 5/3462

(57) ABSTRACT

The invention relates to compounds of the formula (1) in which R₁ and R₂ respectively are a sterically hindered cyclic amine and their use as additive for increasing the notched impact resistance of polyetherketones.

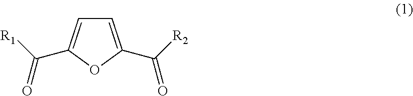

The invention relates to chemical compounds derived from furan chemically bonded by way of at least one ester bond or amide bond to at least one sterically hindered amine. The invention also describes a process for the preparation of said compounds.

Sterically hindered cyclic amines are widely used in industry. Starting materials of particular interest are those such as isophthalic acid and aromatic derivatives, these being used for the preparation of N,N′-bis(2,2,6,6-tetramethyl-4-piperidinyl)-1,3-benzenedicarboxamide, or of other sterically hindered cyclic amine derivatives. 4-Amino-2,2,6,6-tetramethylpiperidine (TAD) is a typical unit of what are known as HALS systems. TAD can be prepared continuously on an industrial scale in accordance with EP0776887 B1.

EP 1556350 B1 describes an optimized process for the preparation of N,N′-bis(2,2,6,6-tetramethyl-4-piperidinyl)-1,3-benzenedicarboxamide with use of certain organic solvents with relatively little pollution of the environment.

However, the aromatic starting materials are always obtained on an industrial scale from xylene, which is a petroleum derivative. Because petroleum resources are finite, it is advantageous to replace this building block with “green” alternatives.

It was therefore an object to provide novel sterically hindered cyclic amines from synthetic building blocks which can at least to some extent be obtained from renewable raw materials. Another object was to provide a process which can prepare said sterically hindered cyclic amines and which is technically simple and environmentally advantageous.

It has been found that, surprisingly, the divalent aromatic carbonyl compound 2,5-furandicarboxylic acid (FDCA) is suitable as building block for sterically hindered piperidine compounds. By use of suitable methods it is possible to obtain FDCA from 5-hydroxymethylfurfural (5-HMF), for example as in

WO 2011/043661 A1 or US 2011/0092720 A1. 5-HMF can be obtained from renewable raw materials.

The invention provides compounds of the formula (1)

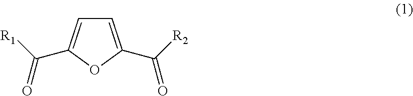

in which R₁ and R₂ respectively are a sterically hindered cyclic amine.

It is preferable that the moieties R₁ and R₂ correspond to the formulae (2a), (2b), and (2c)

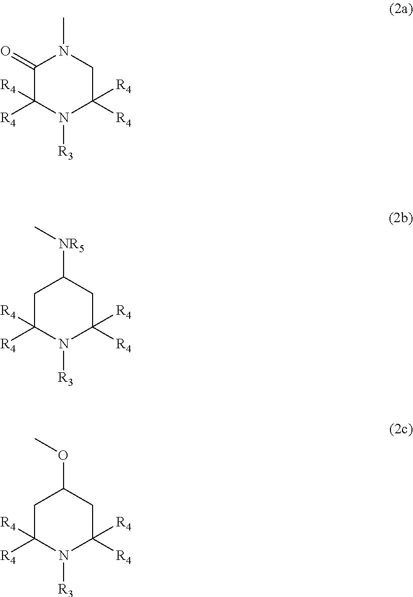

in which
R₃ are H, C₁—O₅-alkyl, or C₁-C₁₀-alkoxy,
R₄ are H, C₁-C₄-alkyl, or C₆-cycloalkyl, and
R₅ are H, or C₁-C₄-alkyl.

It is preferable that R₃ is H or C₁-C₂-alkyl, in particular H.

It is preferable that R₄ is C₁-C₂-alkyl, in particular methyl.

It is preferable that R₅ is H or methyl, in particular H.

Of particular interest are compounds of the formula (1) in which R₁ and R₂ are identical or different and are a moiety of the formula (2d), (2e), or (2f)

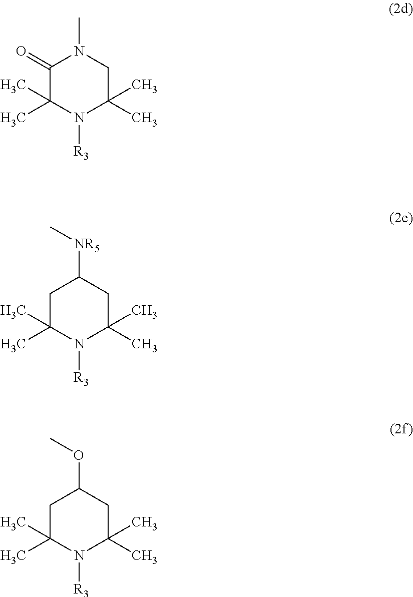

in which R₃ and R₅ are as defined above.

Of very particular interest are compounds of the formula (1) in which R₁ and R₂ are identical or different and are a moiety of the formula (2e) or (2f), for example the compounds N,N′-bis(2,2,6,6-tetramethyl-4-piperidinyl)-2,5-furandicarboxamide and N,N′-bis(2,2,6,6-tetramethyl-4-piperidinyl) 2,5-furandicarboxylate.

The invention further provides a process for the preparation of compounds of the formula (1) via condensation of 2,5-furandicarbonyl dichloride with two equivalents of sterically hindered cyclic amine of the formula H—R₁ and/or H—R₂. It is advantageous to use the amine in 1.8- to 5-molar excess, preferably 2.0- to 3-molar excess, based on 2,5-furandicarbonyl dichloride. The reaction advantageously takes place at a temperature of from 0 to 150° C. In a preferred method, the amine is dispersed or dissolved in a nonpolar or polar organic solvent, for example hexane, cyclohexane, N-methyl-2-pyrrolidone, tetrahydrofuran, or 1,4-dioxane, the 2,5-furandicarbonyl dichloride is admixed at a temperature of from 0 to 20° C., the mixture is then heated to from 30 to 150° C., and then the 2,5-furandicarboxamide is isolated. Analogously, when the corresponding alcohol H-(2f) is used the 2,5-furandicarboxyl diester is isolated.

The preparation process described above has the disadvantage that it proceeds from acyl chlorides, these being prepared on an industrial scale via reaction of the acids with chlorinated agents such as thionyl chloride. Although acyl chlorides have high reactivity and correspondingly enable achievement of high product yields in short reaction times, acyl chlorides are difficult to handle and are susceptible to hydrolysis if they come into contact with water.

An alternative preparation process has therefore been developed which starts from the dicarboxylic esters and replaces the alcohol with the sterically hindered cyclic amine and, respectively, the corresponding alcohol, and is likewise provided by the invention. In chemical terms, this approach is particularly elegant because the reaction equilibrium can be shifted in the direction of the desired product via distillative removal of the replaced alcohol, for example ethanol or butanol.

The invention therefore further provides a process for the preparation of compounds of the formula (1) via aminolysis or transesterification of dialkyl 2,5-furandicarboxylate with two equivalents of the compounds of the formula H—R₁ and/or H—R₂ in the presence of a metal alkoxide catalyst, for example alkali metal alkoxide catalyst, alkaline earth metal alkoxide catalyst, or transition metal alkoxide catalyst. It is advantageous to use the compound of the formula H—R₁ and/or H—R₂ in 1.8- to 5-molar excess, preference being given to 2.0- to 3-molar excess, based on dialkyl 2,5-furandicarboxylate. Preferred ester is a di-C₁-C₆-alkyl 2,5-furandicarboxylate, in particular di-C₁-C₄-alkyl 2,5-furandicarboxylate, e.g. dimethyl 2,5-furandicarboxylate, diethyl 2,5-furandicarboxylate, or di-n-butyl 2,5-furandicarboxylate.

The molar quantity used of the metal alkoxide catalyst is advantageously from 0.1 to 20 mol %, preferably from 0.2 to 10 mol %, particularly preferably from 0.5 to 5 mol %, based in each case on the molar quantity used of the compound(s) of the formula H—R₁ and/or H—R₂. Particular preference is given to sodium methoxide, potassium methoxide, and titanium(IV) butoxide.

In an advantageous method for the reaction, the reactants and the catalyst are mixed together and heated above the melting point of the reaction mixture, preferably to a temperature of from 60 to 200° C., with distillative removal of the alcohol liberated. The reaction can also be carried out advantageously in an organic solvent with boiling point above that of the alcohol liberated during the reaction, for example xylene. The reaction temperature here should be selected in such a way that it remains at least for a time below the boiling point of the solvent in order that the material removed by distillation during the reaction is the alcohol, rather than the actual solvent.

### Use of the Compounds of the Invention

The compounds of the invention can advantageously be used in polyetherketones, preferably in polyetheretherketones, as additives for improving the ease of coloring of these by pigments, and at the same time improving notched impact resistance.

### Experimental Section

Determination of Charpy impact properties of PEEK with and without additives based on the compounds of the invention, including comparison with Nylostab S-EED.

Charpy notched impact resistance is the impact energy absorbed when a notched test specimen fractures, divided by the initial cross-sectional area of the test specimen at the notch side. Procedure as in DIN EN ISO 179-1.

### Preparation of Test Specimens

The test specimens of the PEEK polymer were prepared from the corresponding pellets by using a heated press to apply a pressure of 50 bar at a temperature of 390° C. The pellets here were applied uniformly to the preheated lower plate within a metal frame of thickness 4 mm (internal dimensions 200×200 mm), and then the polymer was exposed in the melt to the abovementioned pressure for 10 min. After slow depressurization and cooling, the sheet, which was visually homogeneous, was cut to give individual smaller rectangular specimen sections of thickness 4 mm, length 80 mm, and width 10 mm. In order to minimize variance, 10 of these rectangular specimen sections were used for each measurement.

Specimens of this type were prepared a) without further additive, b) with 0.4% by weight of Nylostab S-EED, and c) with 0.4% by weight of an additive of the invention.

### Pendulum Impact Test

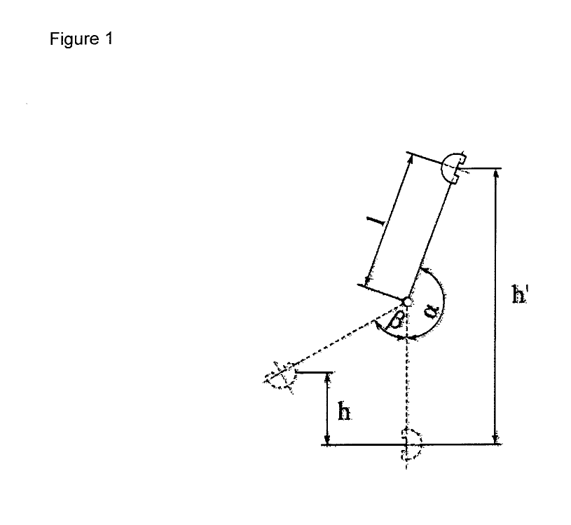

The notched impact resistance tests, which allow conclusions to be drawn concerning the behavior of the plastics specimen on exposure to short-term mechanical impact stress, used a 3/76-50 pendulum impact tester from Feinmechanik Kögel, Leipzig (FIG. 1).

The conditions for the 16-hour conditioning of the specimens and of the measurements were: 23° C. and 50% rel. humidity.

Once the pendulum impact tester had been switched on, the respective current measurement parameters were input, alongside the batch numbers. At the same time, the computer equipped with specific FRK software, was started. A zero-point adjustment was then carried out, care being taken here that the pendulum was motionless. The selection and adjustment of the pendulum here was moreover such that the energy W was in the range from 10 to 80% of the energy that could be provided by the pendulum. The notched impact energy W in kilojoules/m² is thus determined for the PEEK polymer at 23° C. in accordance with the following equation:

W=m·g·(h′−h)
W: notched impact energy in kJ/m²
m: mass of pendulum 0.8 kg
g: acceleration due to gravity (earth: 9.81 m/s²)
h′−h: drop height−pendulum lift height

The actual measurement was made placing the abovementioned test specimens individually in the center of the specimen holder, closing the safety door, and releasing the pendulum, whose notch impacting the specimen had an angle of 45° (depth 2 mm, width 4 mm). The notch on the specimen was on the side facing away from the pendulum. The manual brake was used to brake the subsequent oscillation of the pendulum. The criterion for failure was macroscopically visible fracture of the specimen on application of a particular notched impact energy. Table 1 shows the results of the individual measurements.

### SYNTHESIS EXAMPLES

### Example 1

10 ml (57.1 mmol; 2.2 eq) of 4-amino-2,2,6,6-tetramethylpiperidine were dissolved at room temperature in 40 ml of N-methylpyrrolidone and cooled to 0° C. 5.0 g (25.9 mmol) of 2,5-furandicarbonyl dichloride were added in a plurality of portions. A finely divided pale precipitate forms in the reaction mixture during the exothermic reaction, and is stirred for one hour at 25° C. and for 4 more hours at 100° C. After cooling, the mixture is added to cyclohexane, and the precipitated product is isolated by filtration, washed repeatedly with cyclohexane, and dried.

Yield: 12.9 g of bispiperidinium dihydrochloride salt.

### Example 2

The free base (bispiperidine) is prepared by dissolving 6.7 g of bispiperidinium dihydrochloride salt from example 1 in warm distilled water, and adjusted to pH 11.5 with 25% by weight aqueous ammonia solution. A colorless precipitate forms and is isolated by filtration and washed with water.

Yield after drying: 4.9 g (11.3 mmol, 84% of theory) of colorless, crystalline, odorless solid; melting point 240° C.; Rf=0.35 (methanol/dichloromethane/NH₃(aq) 3:1:0.01);

Solubility: soluble in DMF, methanol, ethanol; insoluble in water, hexane

Elemental analysis: found: C, 65.7; H, 9.7; N, 12.7.
theor. C₂₄H₄₀N₄O₃: C, 66.7; H, 9.3; N, 13.0.

### Example 3

5 g (23.6 mmol) of diethyl 2,5-furandicarboxylate are dissolved in 47 ml of xylene (mixture of the isomers) with stirring, and 9 ml (51.4 mmol; 2.2 eq) of 4-amino-2,2,6,6-tetramethylpiperidine are added. The mixture is heated to 60° C. and 2.5 ml of a 30% by weight sodium methanolate solution in methanol are added. The mixture is heated for 7 hours to 110° C., some of the alcohol being removed by distillation here. Concentration of the mixture by evaporation to dryness in vacuo gives a yellowish powder, which is triturated with ethyl acetate, filtered, and dried. This gives 6.9 g of colorless crystalline powder.

### Example 4

5.3 g (25.0 mmol) of diethyl 2,5-furandicarboxylate, 8.7 ml (49.7 mmol; 2.0 eq) of 4-amino-2,2,6,6-tetramethylpiperidine, and 320 mg of sodium methanolate powder are weighed into a sealable glass reactor, flushed with argon, and heated for 2 hours at 150° C. in a microwave synthesis reactor (Monowave 300, Anton Paar). The resultant product is cooled, dissolved in dichloromethane, washed with aqueous 1-molar NaOH solution, and dried.

### Example 5

5 g (18.6 mmol) of dibutyl 2,5-furandicarboxylate, 10 ml (57.1 mmol; 3.1 eq) of 4-amino-2,2,6,6-tetramethylpiperidine, and 300 mg of sodium methanolate powder are charged to a glass flask with superposed microdistillation system, flushed with argon, and heated for 5 hours to 200° C., some of the alcohol being removed by distillation here. After cooling, the crude product is triturated with diethyl ether, filtered, and dried. Yield 5.7 g of crystalline product.

### Example 6

5 g (18.6 mmol) of dibutyl 2,5-furandicarboxylate and 7.8 ml (44.6 mmol; 2.4 eq) of 4-amino-2,2,6,6-tetramethylpiperidine are dissolved in 15 ml of xylene (isomer mixture) in a glass flask with superposed microdistillation system, and 300 mg of sodium methanolate powder are added. The reaction mixture is heated for 10 hours to 130° C., some of the alcohol liberated being removed by distillation here. Finally, the solvent is also removed by distillation in vacuo. After cooling, the crude product is triturated with diethyl ether, filtered, and dried. Yield 6.7 g of crystalline product, R_{f}=0.1 (methanol).

IR (ATR, powder): \tilde{v}/cm⁻¹=3331 (w), 3294 (w), 2961 (m), 2917 (w), 1672 (s), 1647 (s), 1635 (s), 1598 (m), 1571 (s), 1526 (m), 1496 (s), 1453 (w), 1375 (m), 1363 (m), 1317 (s), 1258 (m), 1241 (m), 1205 (m), 1114 (w), 1010 (m), 822 (s), 763 (m), 693 (m), 602 (m).

¹H NMR (d₆-DMSO, 400 MHz): δ/ppm=1.05 (12H, s), 1.14-1.20 (4H, t, J=12 Hz), 1.16 (12H, s), 1.68-1.72 (4H, dd, J=4 Hz, J=12 Hz), 4.19-4.29 (2H, m, J=4 Hz, J=8 Hz, J=12 Hz), 7.11 (2H, s), 8.01 (2H, d, J=8 Hz); ¹³C NMR (d₆-DMSO, 100 MHz): δ/ppm=28.6, 34.6, 42.0, 44.1, 50.4, 114.4, 148.3, 156.4.

### Example 7

10.1 g (37.6 mmol) of dibutyl 2,5-furandicarboxylate, 15.1 g (95.6 mmol; 2.5 eq) of 4-hydroxy-2,2,6,6-tetramethylpiperidine, and 300 μl of tetrabutyl titanate are heated to 150° C. in a glass flask with superposed distillation system under a stream of nitrogen at low flow rate, with stirring, to give a clear melt. Removal of butanol by distillation rapidly begins. Within a period of 4 hours, the temperature is increased to 180° C. After cooling, the cooled melt is taken up in toluene, and the titanium compound obtained is precipitated hydrolytically. Finally the solvent is removed by distillation in vacuo. The crude product is purified chromatographically.

R_{f}=0.84 (methanol/dichloromethane 3:1).

The product derived from dimethyl 2,5-furandicarboxylate can be prepared analogously.

### Example 8

5 g (27.2 mmol) of dimethyl 2,5-furandicarboxylate are dissolved in 50 ml of xylene, with stirring, and 9.3 g (59.8 mmol; 2.2 eq) of 3,3,5,5-tetramethyl-2-piperazinone are added. The mixture is heated to 60° C., and 2.5 ml of a 30% by weight sodium methanolate solution in methanol are added. The mixture is heated for 5 hours to from 110 to 120° C., and methanol is removed by distillation here. Concentration by evaporation to dryness in vacuo gives a yellowish powder, which is triturated with ethyl acetate, filtered, and dried.

### Usage Examples:

PEEK=polyetheretherketone from the monomer 4-hydroxyphenyl(4-phenoxyphenyl)methanone
Nylostab® S-EED=N,N′-bis(2,2,6,6-tetramethyl-4-piperidinyl)-1,3-benzenedicarboxamide (Clariant)

### Extrusion of Polyetheretherketone (PEEK)

### A. Drying

The residual moisture contents of production batches of the commercially available PEEK Vestakeep® 2000G (producer: Evonik) are up to about 0.5% by weight. It is therefore advisable to predry the polymer in powder form at 160° C. for about 4 hours in a convection drying oven or in an evacuatable drying oven to residual moisture levels of <0.02% by weight.

### B. Extrusion

A Leistritz ZSE 27 HP twin-screw extruder, 44D, with 3 heating zones, was used to process the PEEK, where appropriate with the corresponding additive. The screw diameter was 30 mm.

The predried PEEK powder was charged by way of a hopper preheated to T=180° C. The temperature in the feed zone of the thermally insulated twin-screw extruder (polished screw made of stainless steel, peripheral velocity of screw 10 m/min., rotation rate 80 rpm) was set at 350° C. The first heating zone was heated to from 350 to 360° C. At the end of the first heating zone, the additive of the invention was metered into the system, likewise by way of a hopper. The commercially available additive Nylostab® S-EED was used at the same concentration for comparison. The quantities were judged to give a final total concentration of 0.4% by weight of additive. As further reference specimen, PEEK was processed without addition of any additive. The second heating zone of the extruder was adjusted to a temperature range from 360 to 370° C., and finally a temperature level of from 370 to 380° C. was reached in the third heating zone. The outlet die was adjusted to a temperature of 390° C. The melt strand was passed into a waterbath by way of drop height of 100 cm, and at the end of the waterbath was comminuted by way of a chopper to give pellets. The pellets were used for the further characterization procedure.

The characterization procedure comprised Charpy notched impact resistance determination in accordance with ISO 179/1eA at T=23° C.:

TABLE 1

| | | Notched impact |
|---|---|---|
| | Additive concentration | resistance |
| Additive from example | [% by weight] | [kJ/m²] |
| None | — | 6.2 |
| Nylostab S-EED (comp.) | 0.4 | 7.8 |
| Example 2 | 0.4 | 9.0 |
| Example 7 | 0.4 | 8.8 |
| Example 8 | 0.4 | 8.5 |

1. A compound of the formula (1) 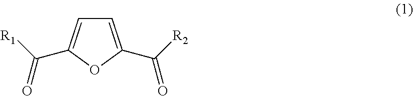
wherein R₁ and R₂ respectively are a sterically hindered cyclic amine.

2. The compound as claimed in claim 1, wherein the moieties R₁ and R₂ correspond to the formulae (2a), (2b), and (2c) 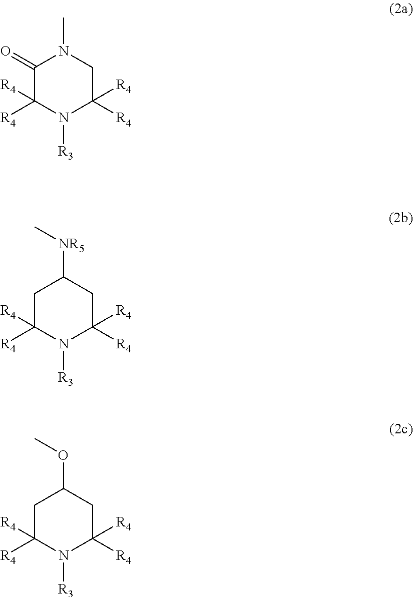
wherein
R₃ are H, C₁-C₅-alkyl, or C₁-C₁₀-alkoxy,
R₄ are H, C₁-C₄-alkyl, or C₆-cycloalkyl, and
R₅ are H, or C₁-C₄-alkyl.

3. The compound as claimed in claim 1, wherein R₃ is H or C₁-C₂-alkyl.

4. The compound as claimed in claim 1, wherein R₄ is C₁-C₂-alkyl.

5. The compound as claimed in claim 1, wherein R₅ is H or methyl.

6. The compound as claimed in claim 5, wherein R₁ and R₂ are identical or different and are a moiety of the formula (2d), (2e), or (2f)

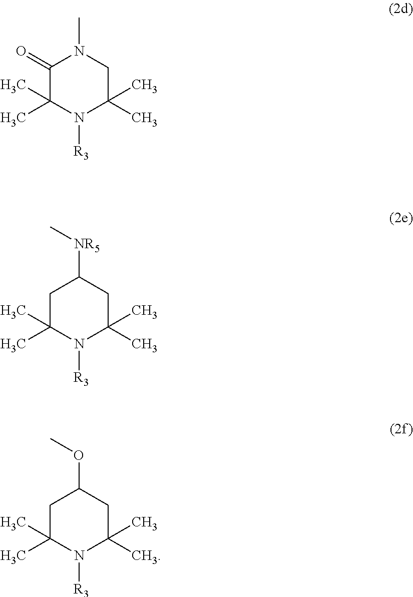

7. The compound as claimed in claim 1, which is N,N′-bis(2,2,6,6-tetramethyl-4-piperidinyl)-2,5-furandicarboxamide or N,N′-bis(2,2,6,6-tetramethyl-4-piperidinyl) 2,5-furandicarboxylate.

8. A process for the preparation of a compound-of the formula (1) 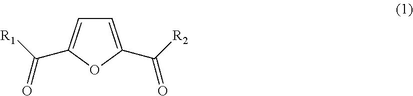
wherein R₁ and R₂ respectively are a sterically hindered cyclic amine, comprising the step of condensing 2,5-furandicarbonyl dichloride with two equivalents of sterically hindered cyclic amine of the formula H—R₁, H—R₂ or a combination thereof.

9. A process for the preparation of a compound of the formula (1) 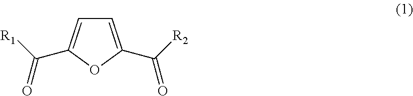
wherein R₁ and R₂ respectively are a sterically hindered cyclic amine,
comprising the step of aminolysis or transesterification of dialkyl 2,5-furandicarboxylate with two equivalents of the compounds of the formula H—R₁, H—R₂ or a combination thereof, in the presence of a metal alkoxide catalyst.

10. A method for increasing the notched impact resistance of a polyetherketone comprising the step of adding a compound of the formula (1) 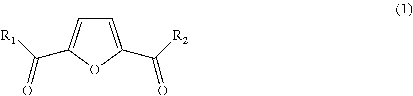
wherein R₁ and R₂ respectively are a sterically hindered cyclic amine, as an additive during the manufacture of the polyetherketone.
